(19) United States
(12) Patent Application Publication
(10) Pub. No.: US 2024/0026271 A1
(43) Pub. Date: Jan. 25, 2024

(54) CELL CULTURE SYSTEM AND CELL CULTURE METHOD

(71) Applicants: CANON KABUSHIKI KAISHA, Tokyo (JP); CANON MEDICAL SYSTEMS CORPORATION, Tochigi (JP)

(72) Inventors: HISAFUMI EBISAWA, Tokyo (JP); ICHIRO HARADA, Kanagawa (JP); YOJI YAMAMOTO, Tokyo (JP)

(21) Appl. No.: 18/350,940

(22) Filed: Jul. 12, 2023

(30) Foreign Application Priority Data
Jul. 22, 2022 (JP) 2022-117226

(51) Int. Cl. C12M 1/12; C12M 1/34

(52) U.S. Cl. CPC C12M 25/14; C12M 41/00

(57) ABSTRACT

There are suitability and unsuitability of a shape of a substrate used for cell culture, for each step in various cell culture protocols, and it has been difficult to continue providing a culture substrate shape suitable for each step. Provided is a cell culture system including: a culture sheet deformable by external force, to which a cell can adhere and be cultured; a die member configured to be pressed against the culture sheet to deform a shape of the culture sheet into a randomly-selected shape; an optical device configured to observe a state of the cell adhering onto the culture sheet; and a drive unit configured to move the die member, to change a randomly-selected portion of the culture sheet into a shape along the die member or release the culture sheet from deformation.

### BACKGROUND OF THE INVENTION

### Field of the Invention

The present invention relates to a cell culture system that uses a deformable culture sheet to which cells can adhere, and to a cell culture method in which a culture sheet can be deformed in each step.

### Description of the Related Art

For culture of adherent cells, typically, a flat substrate such as a petri dish is used, and many petri dishes are required to culture a large amount of cells. In Japanese Patent Application Laid-Open No. 2019-141034, there is disclosed a culture method in which cells are caused to adhere to a three-dimensional structure such as a fiber and are cultured, to increase an amount of cells per unit volume. The culture method of Japanese Patent Application Laid-Open No. 2019-141034 allows an enzyme solution used during detachment to easily permeate into cells adhering to a convex surface, and hence has an advantage in enabling easier detachment of cells.

In Japanese Patent No. 6806055, there is disclosed a cell culture device that deforms a flexible sheet, to thereby provide a culture substrate suitable for culture of a target cell. In the device of Japanese Patent No. 6806055, a sheet is pressed against a die member to form a plurality of recesses as wells in the flat sheet, and many spheroids of cells are cultured in the wells. When this method is adopted to a sheet to which cells can adhere, during seeding and culture, cells remain in predetermined places due to the recesses formed in the sheet, and hence seeding is easy. Further, a culture area per unit volume can be increased. Moreover, the sheet is returned to a flat state during close observation of the cells, and hence focusing on the entire observation area can be achieved.

### SUMMARY OF THE INVENTION

Meanwhile, according to the method of Japanese Patent Application Laid-Open No. 2019-141034, it is difficult to achieve uniform focusing on the entire area of the substrate in observing cells with a microscope, and there arises a problem of incapability of precisely observing the shapes of cells or colonies. Further, cells are unlikely to be caught in the fiber, and there arises another problem of presence of some cells that come off the fiber during seeding and fail to be cultured.

Further, according to the culture method of Japanese Patent No. 6806055, the die member cannot be moved. Thus, a position where deformation is caused on the sheet cannot be adjusted. That is, according to the culture method of Japanese Patent No. 6806055, the sheet cannot be deformed, or the shape of the sheet cannot be changed into a different shape (from convex to concave, or the like), so as to suit a target cell, and a sheet shape suitable for each step in a protocol cannot be provided to the cell.

The present invention has been devised in view of the above-described problems. That is, an object of the present invention is to provide a cell culture system and a cell culture method for appropriately deforming a culture substrate into a shape suitable for each step in various cell culture protocols.

According to one embodiment of the present invention, there is provided a cell culture system including: a culture sheet which is deformable, and to which a cell is allowed to adhere; a die member configured to be pressed against the culture sheet to deform the culture sheet; a drive unit configured to drive the die member; and an optical-image acquisition unit configured to acquire an optical image of the cell.

Further, according to another embodiment of the present invention, there is provided a cell culture method including: seeding a cell onto a culture sheet which is deformable and to which the cell is allowed to adhere; culturing the cell; observing the cell; and detaching the cell. At least one of the seeding, the culturing, the observing, or the detaching includes pressing a die member to deform at least a part of the culture sheet.

Further features of the present invention will become apparent from the following description of exemplary embodiments with reference to the attached drawings.

### BRIEF DESCRIPTION OF THE DRAWINGS

FIG. 1A is a view for illustrating a cell culture system according to an embodiment of the present invention.

FIG. 1B is a view for illustrating the cell culture system according to the embodiment of the present invention.

FIG. 1C is a view for illustrating the cell culture system according to the embodiment of the present invention.

FIG. 2A is a view for illustrating an example in which a vessel accommodating a culture sheet is included.

FIG. 2B is a view for illustrating the example in which the vessel accommodating the culture sheet is included.

FIG. 2C is a view for illustrating the example in which the vessel accommodating the culture sheet is included.

FIG. 3A is a view for illustrating an example in which a vessel is included and two or more die members are further included.

FIG. 3B is a view for illustrating the example in which the vessel is included and the two or more die members are further included.

FIG. 3C is a view for illustrating the example in which the vessel is included and the two or more die members are further included.

FIG. 4A is a view for illustrating an example in which the culture sheet is a part of the vessel.

FIG. 4B is a view for illustrating the example in which the culture sheet is a part of the vessel.

FIG. 4C is a view for illustrating the example in which the culture sheet is a part of the vessel.

FIG. 5A is a view for illustrating an example in which the culture sheet is a part of the vessel and two or more die members are further included.

FIG. 5B is a view for illustrating the example in which the culture sheet is a part of the vessel and the two or more die members are further included.

FIG. 5C is a view for illustrating the example in which the culture sheet is a part of the vessel and the two or more die members are further included.

FIG. 6 is a view for illustrating an example in which a drive unit is included in the vessel.

FIG. 7 is a view for illustrating an example in which an outer vessel is included.

FIG. 8 is a view for illustrating an example in which an external-force applying unit is included.

FIG. 9 is a view for illustrating an example in which an external-force applying unit is included and two or more die members are further included.

FIG. 10A is a view for illustrating an example in which the way of pressing a die member including a recess varies depending on the presence or absence of the external-force applying unit.

FIG. 10B is a view for illustrating the example in which the way of pressing the die member including the recess varies depending on the presence or absence of the external-force applying unit.

FIG. 10C is a view for illustrating the example in which the way of pressing the die member including the recess varies depending on the presence or absence of the external-force applying unit.

FIG. 11A is a view for illustrating a manner in which the culture sheet is deformed along a die member including projections and recesses by the external-force applying unit.

FIG. 11B is a view for illustrating the manner in which the culture sheet is deformed along the die member including projections and recesses by the external-force applying unit.

FIG. 12A is a view for illustrating deformation of the entire culture sheet by press of the die member against only a part of the culture sheet.

FIG. 12B is a view for illustrating deformation of the entire culture sheet by press of the die member against only a part of the culture sheet.

FIG. 12C is a view for illustrating deformation of the entire culture sheet by press of the die member against only a part of the culture sheet.

FIG. 13 is an explanatory view for illustrating Example 1.

FIG. 14 is an explanatory view for illustrating Example 2.

FIG. 15A is an explanatory view for illustrating the die member.

FIG. 15B is an explanatory view for illustrating the die member.

FIG. 15C is an explanatory view for illustrating the die member.

FIG. 15D is an explanatory view for illustrating the die member.

FIG. 15E is an explanatory view for illustrating the die member.

FIG. 15F is an explanatory view for illustrating the die member.

FIG. 15G is an explanatory view for illustrating the die member.

FIG. 15H is an explanatory view for illustrating the die member.

FIG. 15I is an explanatory view for illustrating the die member.

### DESCRIPTION OF THE EMBODIMENTS

### First Embodiment

As a first embodiment of the present invention, there is provided a cell culture system including: a culture sheet which is deformable, and to which a cell is allowed to adhere; a die member configured to be pressed against the culture sheet to deform the culture sheet; a drive unit configured to drive the die member; and an optical-image acquisition unit configured to acquire an optical image of the cell.

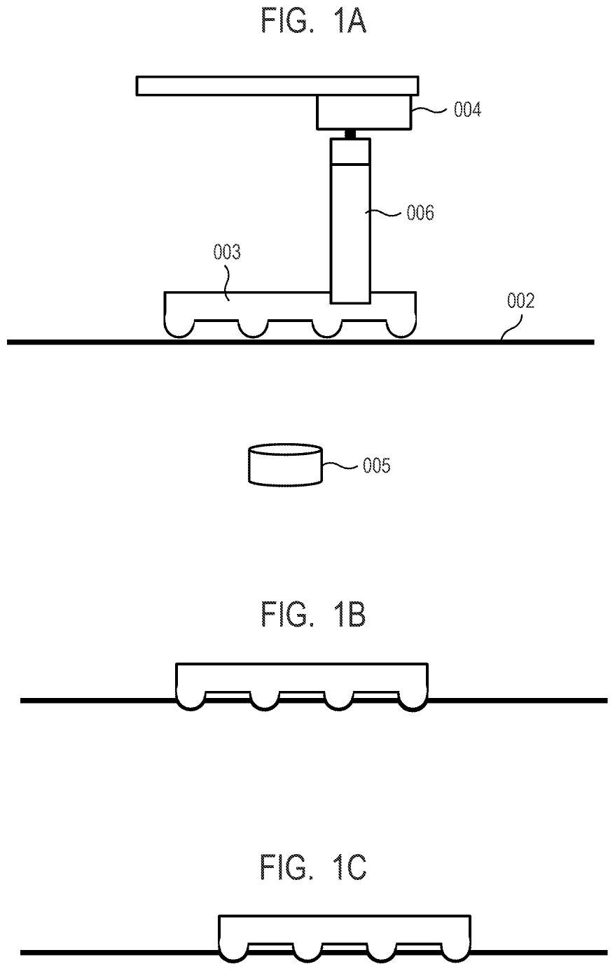

The cell culture system according to the first embodiment is exemplified in FIG. 1A to FIG. 1C. The cell culture system includes a deformable culture sheet 002 to which cells can adhere. The cell culture system further includes a die member 003, a drive unit 004, and an optical-image acquisition unit 005. An arm 006 illustrated in FIG. 1A may be omitted, and the drive unit 004 may come into direct contact with the die member 003. The die member 003 has a shape corresponding to a target shape in order to deform the culture sheet 002 into the target shape. The culture sheet 002 is deformed by press of the die member 003 against the culture sheet 002 (FIG. 1B). The die member 003 can be moved by the drive unit 004 and can deform a randomly-selected portion of the culture sheet 002. Further, the optical-image acquisition unit 005 for observing a state of cells adhering to the culture sheet 002 is placed. Based on information about an acquired optical image, the drive unit 004 can drive the die member 003 and change the shape of the culture sheet 002 in accordance with the state of the cells.

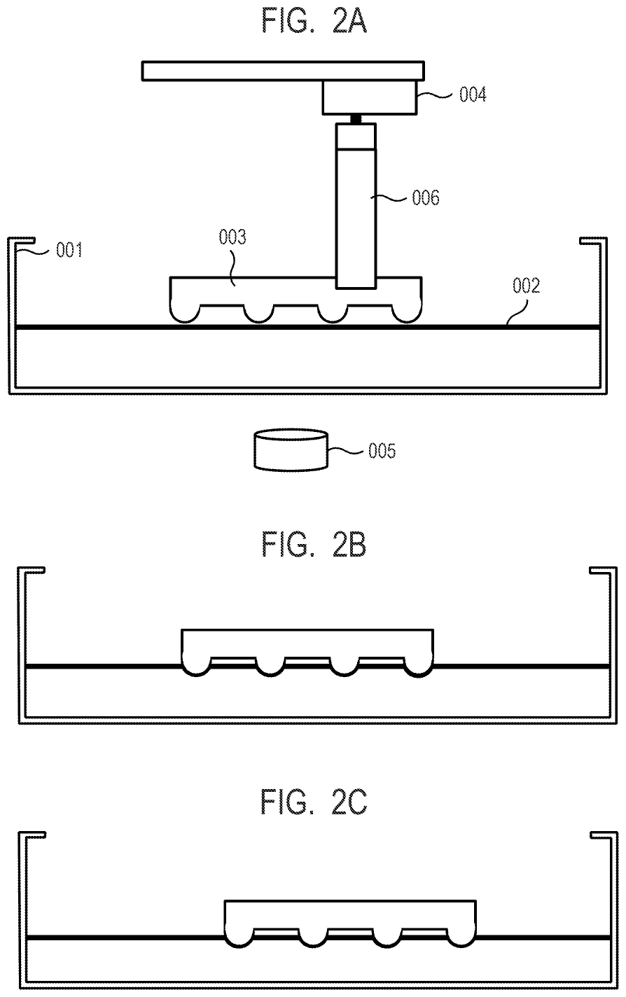

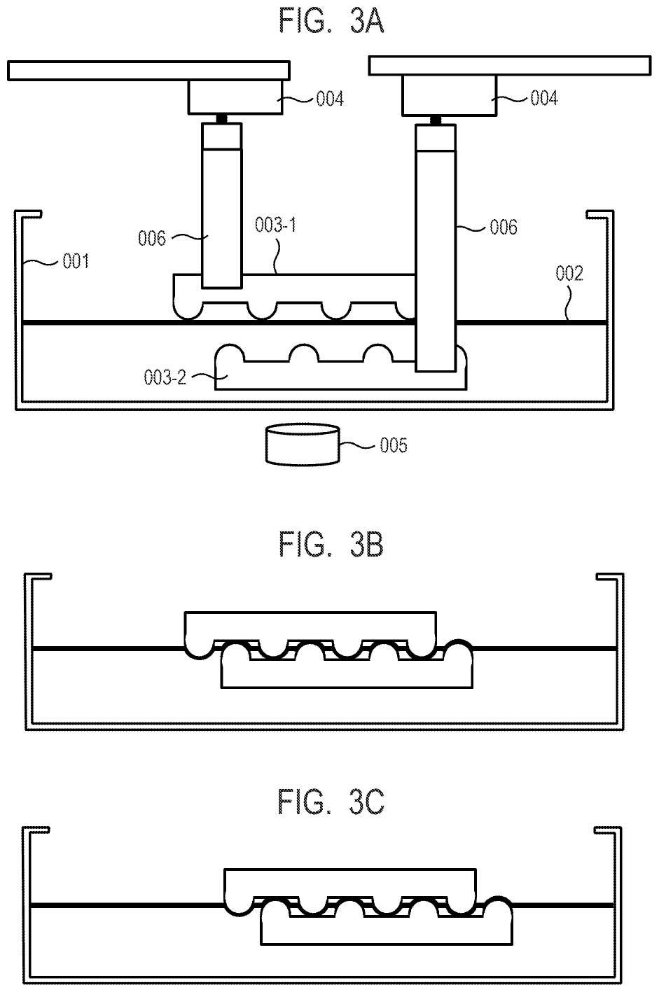

The cell culture system of the first embodiment can further include a vessel 001 accommodating a culture medium and the culture sheet 002 as illustrated in FIG. 2A to FIG. 2C. Moreover, the cell culture system of the first embodiment can include two or more die members 003 including a first die member 003-1 and a second die member 003-2 as illustrated in FIG. 3A to FIG. 3C. The first die member 003-1 is pressed against a culture surface of the culture sheet 002, and the second die member 003-2 is pressed against a back surface opposite to the culture surface of the culture sheet 002.

Note that, in the culture sheet 002, a surface to which cells adhere during culture is sometimes referred to as a culture surface, and a surface opposite thereto is sometimes referred to as a back surface in the present specification. Usually, out of two surfaces of the sheet, a surface facing upward is a culture surface, and a surface facing downward is a back surface.

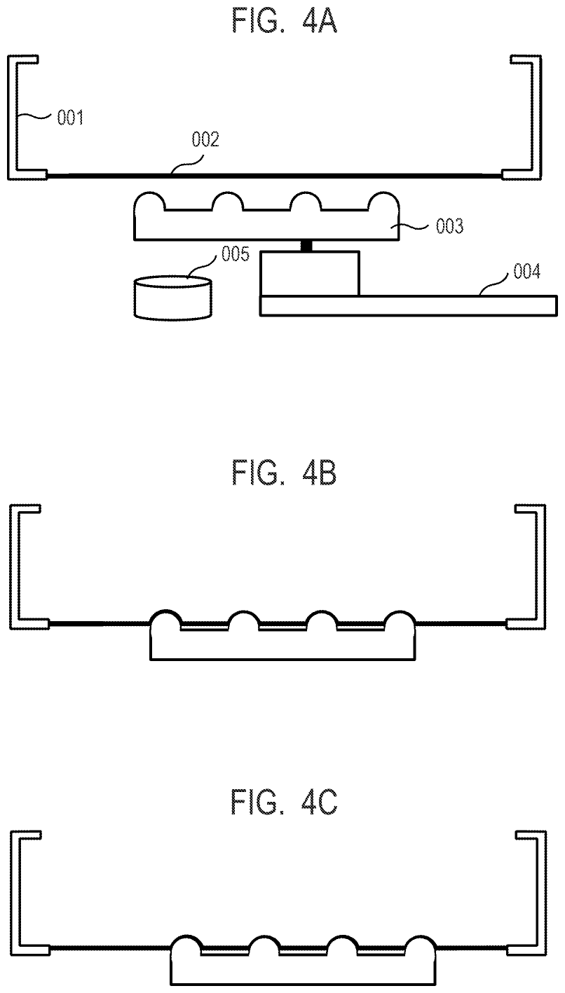

As illustrated in FIG. 4A to FIG. 4C, the cell culture system of the first embodiment can include the vessel 001 accommodating the culture medium (the culture medium is not shown), and the culture sheet 002 can form a part of the vessel 001.

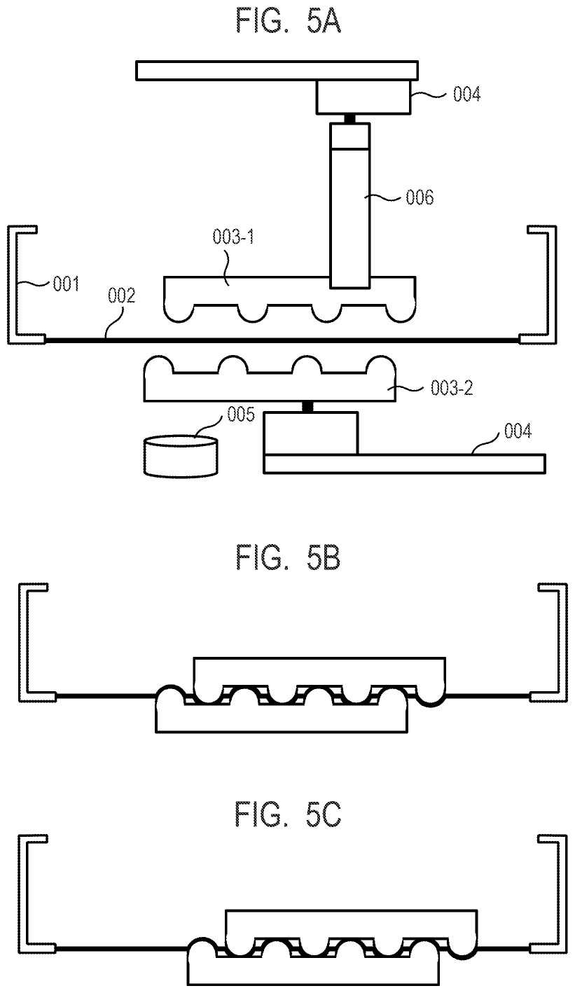

Further, as illustrated in FIG. 5A to FIG. 5C, the cell culture system of the first embodiment can include the vessel 001 accommodating the culture medium (the culture medium is not shown), the culture sheet 002 can form a part of the vessel 001, and the cell culture system can include two or more die members 003 including the first die member 003-1 and the second die member 003-2. In this case, the first die member 003-1 is pressed against the culture surface of the culture sheet 002, and the second die member 003-2 is pressed against the back surface opposite to the culture surface of the culture sheet 002.

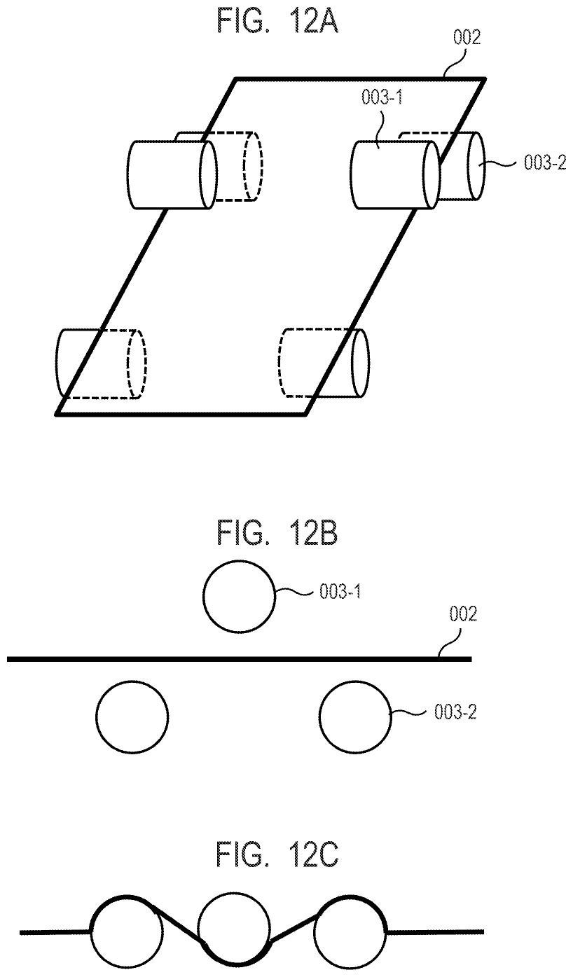

In the cell culture system of the first embodiment, as illustrated in FIG. 12A to FIG. 12C, the die member 003 can be prevented from coming into contact with a partial area of the culture surface of the culture sheet 002.

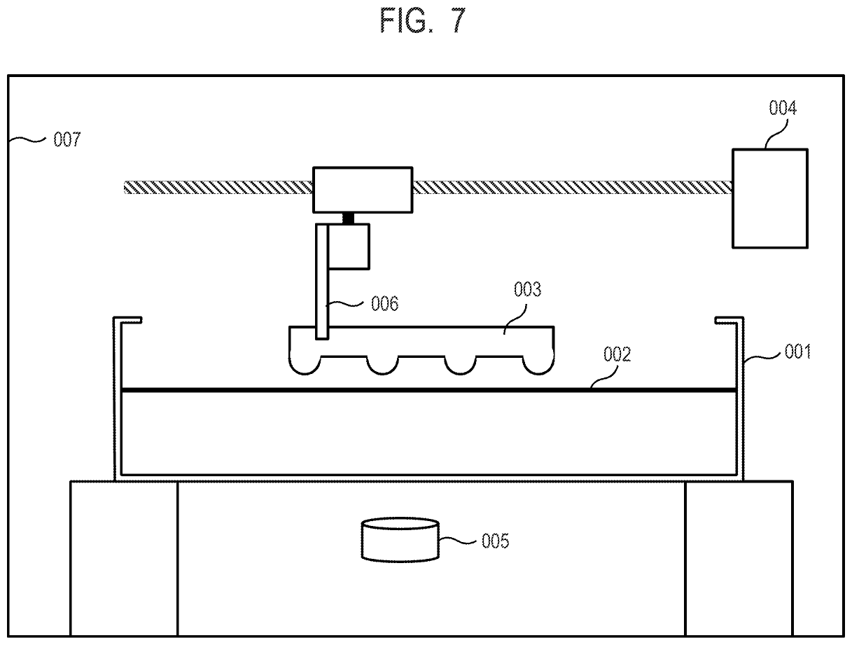

As illustrated in FIG. 7, the cell culture system of the first embodiment can further include an outer vessel 007 accommodating the vessel 001, the culture sheet 002, the die member 003, and the drive unit 004.

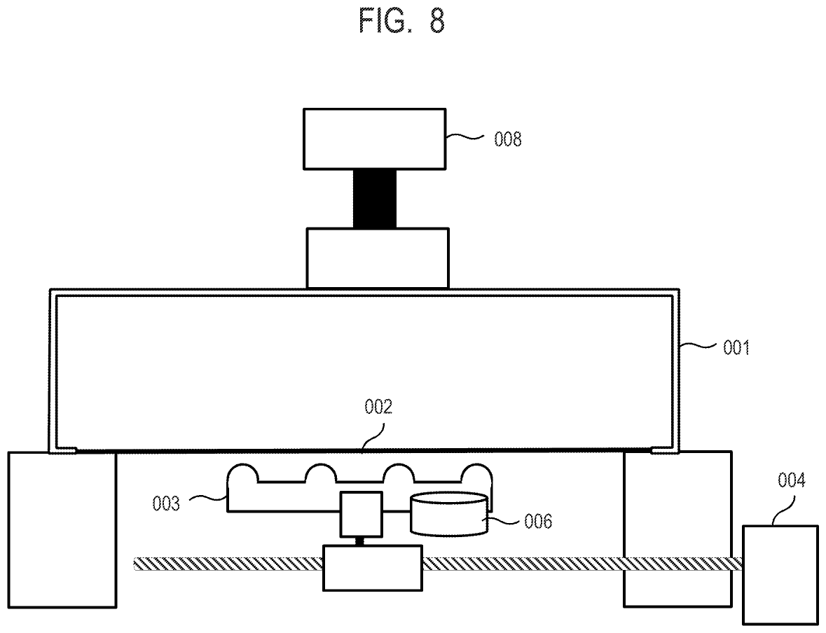

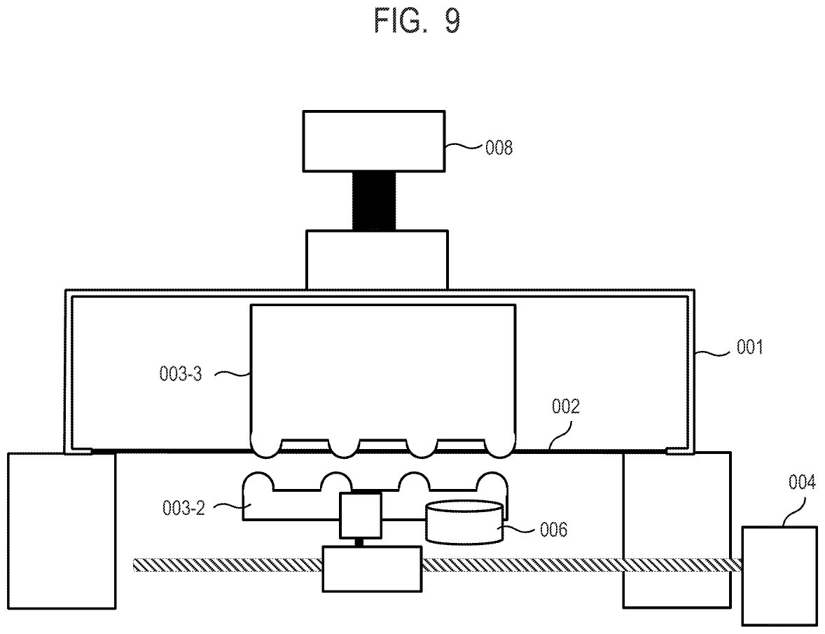

Further, the cell culture system of the first embodiment can include an external-force applying unit 008 that is distinct from the drive unit 004 and is configured to press the die member 003 against the culture sheet 002. In FIG. 8, there is illustrated an example in which the external-force applying unit 008 applies external force to the vessel. At that time, as illustrated in FIG. 9, an additional die member 003-3 may be provided also in the vessel. The additional die member 003-3 is not connected to the drive unit 004 and is pressed against the culture surface of the culture sheet 002 by the external-force applying unit 008. Meanwhile, the external-force applying unit 008 is not limited to the example illustrated in FIG. 8, and may be, for example, one that applies external force by increasing and reducing the pressure in the vessel.

Further, the cell culture system of the first embodiment can include a drive control unit for controlling the drive unit 004, and the drive control unit can control the drive unit based on an optical image of a cell such that at least a part of the culture sheet has any of a flat shape, a concave shape, and a convex shape. The drive control unit preferably has a function as a computer that performs arithmetic operations and storage, can include a central processing unit (CPU), a random access memory (RAM), a read only memory (ROM), and a hard disk drive (HDD), and can further include a communication interface (I/F), a display device, and an input device.

### Vessel and Culture Sheet

The culture system of the first embodiment may include the vessel 001 accommodating the culture medium and the culture sheet 002 (FIG. 2A to FIG. 2C). As another example, the culture system may include the vessel 001 accommodating the culture medium, and the culture sheet 002 may form a part of the vessel (FIG. 4A to FIG. 4C). Note that, the culture medium is not shown in each drawing.

As a material of the vessel 001, resin, glass, and the like that can be easily processed and observed and are optically transparent are suitable. For resin, polystyrene, polycarbonate, acryl, and the like can be used. Further, the material is not limited to one having transparency as long as it does not block an optical path during observation. In the case of a large vessel in particular, use of metal or the like for the vessel is effective also in terms of strength.

From the viewpoint of preventing external contamination with foreign matters and ensuring the temperature or the humidity, the vessel 001 is preferably configured such that a lid can be attached thereto. Further, in a case in which replacement of the culture medium or the like is manually performed, an opening is preferably wide. On the other hand, in a case in which water supply and drainage through a tube are performed, it is effective to narrow the opening and provide a supply/discharge port into which a tube can be inserted, from the viewpoint of preventing contamination.

In the example illustrated in FIG. 2A to FIG. 2C, preferably, the culture sheet 002 is fixed to the inside of the vessel. In a case in which it is difficult to fix the culture sheet 002 directly to the vessel, a frame member capable of fixing the culture sheet 002 is prepared, and a configuration in which the frame member can be placed in the vessel is effective also. In this case, a configuration in which fixing can be achieved by fitting of the frame member into the vessel is preferred. Further, considering an amount of deformation to be caused by the die member 003, the culture sheet 002 is preferably fixed to the vessel or the frame member with some slack therein.

The configuration illustrated in FIG. 4A to FIG. 4C corresponds to a system in which the culture sheet 002 forms a part of the vessel 001 as illustrated therein.

In a case in which the culture sheet 002 and the vessel 001 are made of different materials, sealing between the culture sheet 002 and the vessel 001 against leakage of a liquid such as the culture medium is required. The sealing can be achieved by fixing with the use of an O ring or the like. As another example, for the sealing, it is also effective to fix with the use of an adhesive or fix by a technique such as welding or bonding without the use of an adhesive.

In an example in which the culture sheet 002 and the vessel 001 are made of the same material, a pouch-shaped culture bag can be used. In this case, it is effective to keep the pressure in the culture bag relatively high in order to maintain a space in the vessel. It is also effective to cover the bag with a cover in order to prevent the bag from being broken by an impact from an external sharp object. For example, a bag formed of the culture sheet 002 and a vessel covered with a cover formed of a resin material can be used.

Further, though the same material is used, materials that have been processed by different methods are used in some cases. For example, in a case in which a resin material such as polycarbonate is used, it is effective to increase the entire thickness of the vessel 001 to maintain the strength thereof while using a thin sheet material for the culture sheet 002 to make the culture sheet 002 deformable.

It is preferred to perform surface treatment such as plasma treatment on the culture sheet 002 in order to allow cells to easily adhere to the culture sheet 002. At that time, it is desired to perform surface treatment on only a portion where cells are desired to adhere. Further, it is also effective to perform no treatment, or perform surface treatment that makes it harder for cells to adhere, on a portion where cells are not desired to adhere.

### Die Member

The die member 003 is a member to be pressed to change the culture sheet 002 into a randomly-selected shape. The die member 003 functions when pressed, and thus is preferably made of a material that is hard enough not to deform in response to application of external force. Further, in order to precisely form a minute shape, a material having good processability is preferred, and resin, glass, metal, ceramic, and the like can be used.

The die member 003 includes a recess or a projection that serves the purpose of deforming the culture sheet 002.

For example, in FIG. 1A to FIG. 1C and FIG. 2A to FIG. 2C, the die member 003 including a plurality of projections is pressed against the culture sheet from the culture surface. As a result, the culture sheet has a shape concave with respect to the culture surface as illustrated in FIG. 1B and FIG. 2C.

In FIG. 4A to FIG. 4C, the die member 003 including a plurality of projections is pressed against the culture sheet 002 from the back surface opposite to the culture surface. In this case, the culture sheet has a shape convex with respect to the culture surface as illustrated in FIG. 4B.

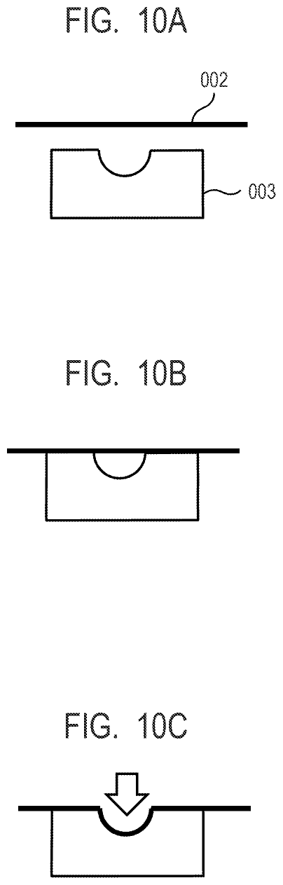

In FIG. 10A, there is illustrated an example in which the die member 003 including a recess is pressed against the culture sheet 002 from the back surface opposite to the culture surface to make the culture sheet concave with respect to the culture surface.

In this example, it is difficult to bring the culture sheet 002 into close contact with the die member 003 as illustrated in FIG. 10B only by pressing the die member 003. By providing an external-force applying unit (not shown) for applying additional external force to the die member 003 as illustrated in FIG. 10C, it is possible to deform the culture sheet 002 along the die member 003. Such an additional external-force applying unit may be, for example, one that reduces the internal pressure of the die member 003 to cause a pressure difference, or pushing of a different die member.

In a case in which a pressure difference is used for the external-force applying unit, implementation is easy in a culture-bag configuration in which the culture sheet 002 forms a part of the vessel 001. For example, by blowing air into a culture bag, or deforming the culture bag to reduce the volume thereof, it is possible to cause a pressure difference.

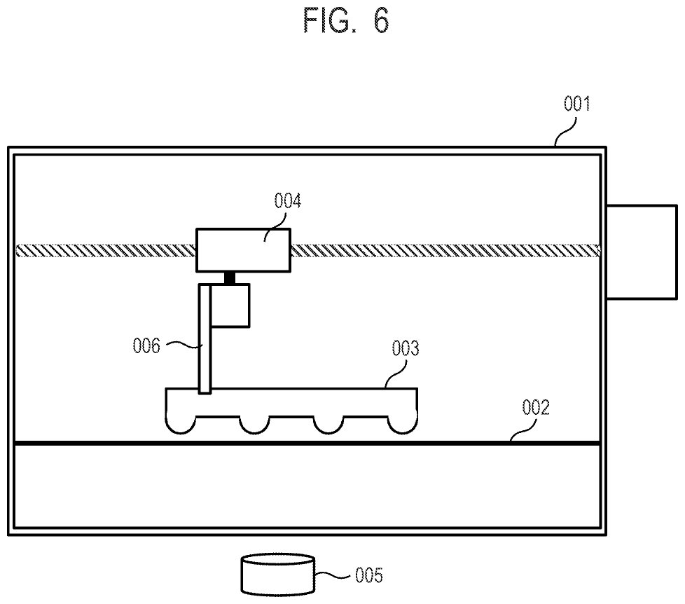

Further, in a case in which the die member 003 as in FIG. 2A to FIG. 2C, FIG. 6, and FIG. 7 is provided only on one side (single side) of the culture surface, a space between the vessel 001 and the culture sheet 002 is enclosed so that the internal pressure of the space is increased when the die member 003 is pressed. The increase of the internal pressure encourages closer contact between the culture sheet 002 and the die member 003, to thereby facilitate deformation of the culture sheet.

Further, in a case in which pushing of the additional die member 003 is used as additional external force, a plurality of die members 003 are prepared and the culture sheet 002 is sandwiched between the die members 003 as illustrated in FIG. 3A to FIG. 3C and FIG. 5A to FIG. 5C, for implementation.

Moreover, in a case in which the die member 003 is provided only on one side (single side) of the surface of the culture sheet 002 (FIG. 1A to FIG. 1C, FIG. 2A to FIG. 2C, FIG. 4A to FIG. 4C, FIG. 6, FIG. 7, and FIG. 8), it is also effective to provide a cushioning member on the other side of the surface of the culture sheet 002. The cushioning member is deformed by press of the die member 003 against it, which serves as external force that presses the culture sheet 002 against the recesses of the die member 003. Note that, as the cushioning member, a transparent gel sheet, a water bag, or the like is preferred, and a transparent gel sheet having a surface to which the culture sheet 002 is stuck can be also used.

Further, in the case illustrated in FIG. 4A to FIG. 4C, filling the culture-surface side of the culture sheet 002 with a culture medium can cause the weight of the culture medium to serve as external force that presses the culture sheet 002 against the recesses of the die member 003.

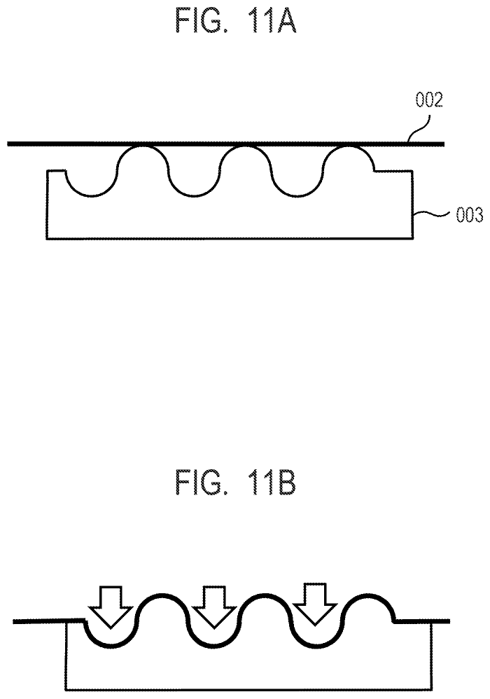

In addition, by using the above-mentioned external force, it is possible to deform the culture sheet 002 as illustrated in FIG. 11B with the use of not only the die member 003 including the recesses, but also the die member 003 including alternate projections and recesses as illustrated in FIG. 11A.

Meanwhile, in some cases, it is not preferred that the culture surface, in particular, in the culture sheet 002, comes into contact with the die member 003. For example, surface treatment of the culture sheet 002 is sometimes spoiled by contact with the die member 003. Further, cells having adhered to the culture sheet 002 are sometimes damaged by contact with the die member.

In such a case, projections and recesses can be formed in the culture sheet 002 by press of the die member 003 against only a part of the culture sheet 002. As illustrated in FIG. 12A, the die members 003 having configurations symmetrical to each other are prepared at opposite ends of the culture sheet 002. FIG. 12B and FIG. 12C are views as seen from the side in FIG. 12A. When the culture sheet 002 is sandwiched from upper and lower sides thereof between the die members 003 as illustrated in FIG. 12B and FIG. 12C, a central portion of the culture sheet 002 not in contact with the die members 003 is also deformed along the die members 003 at the opposite ends. In the case illustrated in FIG. 12A to FIG. 12C, the central portion of the culture sheet 002 not in contact with the die members 003 can be also deformed to form a part of projections and recesses as in FIG. 12C.

Further, it is also possible to perform surface treatment that allows cells to easily adhere to only a portion in which cells are desired to be cultured without contact with the die member 003. Moreover, surface treatment that makes it harder for cells to adhere may be performed on a portion that is to come into contact with the die member 003.

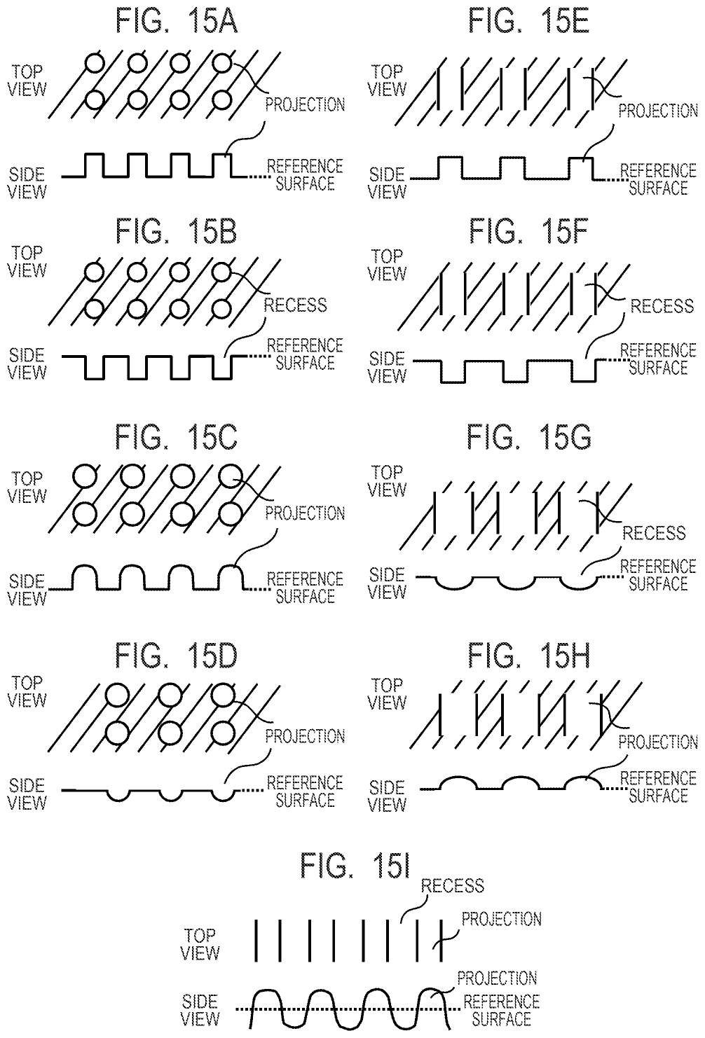

### Shape of Die Member and Projections and Recesses

Whereas the shape of the die member 003 is not limited to any particular shape, the following examples can be given. Specifically, as the die member, one that includes projections or recesses in a dot pattern (FIG. 15A to FIG. 15D), or one that includes bar-shaped projections or bar-shaped recesses (FIG. 15E to FIG. 15I) can be exemplified.

In a case in which the die member includes projections or recesses in a dot pattern, the shape of a bottom surface of each projection or each recess is not limited to any particular shape. The projections or the recesses can each have a diameter of from 1 mm to 5 cm at a bottom surface thereof, can each have a height (depth) of from 1 mm to 1 cm, and can have an interval therebetween of from 5 mm to 20 cm.

In a case in which the projections or the recesses are bar-shaped, the projections or the recesses can each have a width of from 5 mm to 5 cm, can each have a height (depth) of from 5 mm to 1 cm, and can have an interval therebetween of from 5 mm to 5 cm. In any case, the projections or the recesses may be formed linearly (FIG. FIG. 15B, FIG. 15E, and FIG. 15F), or may be formed of a curved surface (FIG. FIG. 15D, FIG. 15G, FIG. 15H, and FIG. 15I).

Note that, in the present specification, projections and recesses are defined with respect to a reference surface of the die member. An example of the reference surface is illustrated in FIG. 15A to FIG. 15I. In a case in which projections and recesses are formed linearly, one of the upper surface and the lower surface of each projection or each recess, whichever has a larger area, is the reference surface (FIG. 15A, FIG. 15B, FIG. 15E, and FIG. 15F). In a case in which either the upper surface or the lower surface of each projection or each recess includes a curved surface, either of the surfaces that does not include a curved surface is the reference surface (FIG. 15C, FIG. 15D, FIG. 15G, and FIG. 15H). In a case in which both of the upper surface and the lower surface of each projection or each recess includes a curved surface, a surface intermediate between the upper surface and the lower surface of each projection or each recess is the reference surface (FIG. 15I). In addition, a portion that projects upward from the reference surface is referred to as a projection or as being convex, and a portion that is recessed downward from the reference surface is referred to as a recess or as being concave.

With regard to the culture sheet, being convex with respect to the culture surface means a shape that is formed by press of the die member including a projection from the side opposite to the culture surface of the culture sheet, or by press of the die member including a recess from the culture surface of the culture sheet. Being concave with respect to the culture surface means a shape that is formed by press of the die member including a recess from the side opposite to the culture surface of the culture sheet, or by press of the die member including a projection from the culture surface of the culture sheet.

Note that, in FIG. 15A to FIG. 15I, there is illustrated a case in which the die member is one component, but the die member may include a plurality of parts, and the parts are not necessarily required to be integral with each other. For example, in FIG. 12A to FIG. 12C, each of the die members 003-1 and 003-2 includes a plurality of rods.

### Drive Unit

The drive unit 004 is used in pressing the die member 003 against the culture sheet 002 or in moving the die member 003 to a randomly-selected position in the culture sheet 002.

By movement of the die member 003, different portions of the culture sheet 002 having been in the state illustrated in each of FIG. 1A, FIG. 2A, FIG. 3A, FIG. 4A, and FIG. 5A can be deformed in accordance with the die member 003 as illustrated in each of FIG. 1B, FIG. 1C, FIG. 2B, FIG. 2C, FIG. 3B, FIG. 3C, FIG. 4B, FIG. 4C, FIG. 5B, and FIG. 5C. As the drive unit 004, any actuator that can precisely move the die member 003 can be used without limitation. For example, a linear stage, a pulse motor with a ball screw, and the like can be used.

Note that, in some cases, it is not preferred that the actuator of the drive unit 004 comes into contact with a liquid such as the culture medium. In such a case, by placing the arm 006 between the drive unit 004 and the die member 003, it is possible to prevent the drive unit 004 from being immersed in the liquid.

A relationship between the size of the vessel 001 and the size of the actuator of the drive unit 004 is important. In a case in which the drive unit 004 is placed in the vessel 001 as illustrated in FIG. 6, there is an advantage in preventing external contamination on one hand, but the vessel 001 becomes more likely to increase in size. Further, the vessel itself is difficult to dispose of, and hence cost associated therewith is likely to increase.

In the system in which the drive unit 004 and the vessel 001 being opened upward are placed in the outer vessel 007 that guarantees a sterile condition as illustrated in FIG. 7, reduction of the size of the vessel 001 and disposability of the vessel 001 can be easily achieved. As the outer vessel 007, a safety cabinet or the like can be used.

Further, as a method in which the drive unit 004 is not placed in the vessel 001, a method of contactless control of the die member 003 with the use of a magnet from outside of the vessel 001 is also effective. In this method, for pushing of the die member 003 into the culture sheet 002, a process of pressurizing the vessel itself from above and below and pressing the die member 003 in the vessel against the culture sheet 002 on the vessel wall surface while slightly distorting the vessel, can be used.

In a case in which the culture sheet 002 forms a part of the vessel 001, the external-force applying unit 008 can be placed outside the vessel as illustrated in FIG. 8. For the actuator, the same one as described above can be used. Further, the vessel 001 is compressed with the use of the external-force applying unit 008, to increase the internal pressure of the vessel, which improves close contact between the die member 003 and the culture sheet 002, to thereby more precisely deform the shape of the culture sheet 002.

Moreover, the culture sheet 002 may be sandwiched between the second die member 003-2 and the additional die member 003-3 from opposite sides as illustrated in FIG. 9. Note that, in a case in which the projections and the recesses of the two die members 003-2 and 003-3 are fit into each other and the same shape is repeatedly formed as in the example illustrated in FIG. 9, on condition that the position of the lower die member 003-2 is determined, the upper die member 003-3 is fit into the position corresponding to the projections and the recesses of the lower die member 003-2 when the upper die member 003-3 is pushed. This eliminates a need of the drive unit 004 for the second die member 003-2 in the system.

### Observation

The optical-image acquisition unit 005 for acquiring optical information indicating a state of a cell adhering onto the culture sheet 002 is placed outside the vessel. As the optical-image acquisition unit 005, various types of microscopes typified by a phase-contrast microscope and cameras can be used.

With the use of the above-described system, a state of a cell is determined from information acquired by an optical device such as the optical-image acquisition unit 005. Then, the drive unit 004 is driven, and a suitable shape of a substrate under the protocol can be provided. The determination as to whether to drive the drive unit 004 may be made based on an operator's check of optical information acquired by the optical-image acquisition unit 005. Further, it is also effective to use a system in which an information processing unit processes optical information and automatically makes determination in accordance with a predetermined algorithm. Moreover, depending on a cell to be cultured or a protocol, there is little variation in progress of cell culture, or a certain degree of deviation in a change timing hardly affects a result of culture, in some cases. In such a case, it is only required to drive the drive unit 004 at a timing determined in advance without using an optical device, and hence the system can be simplified.

### Cell

The cell culture system according to the first embodiment is used for a cell that adheres to a base material to be proliferated. A cell that adheres to a base material to be proliferated includes a so-called adherent cell that continues proliferating while adhering to a base material, and a cell that continues proliferating while floating in a culture medium after adhering to a base material for a certain period of time.

Specific examples of such a cell include an ES cell, an iPS cell, a mesenchymal stem cell, a CHO cell, and an HEK 293 cell.

### Second Embodiment

As a second embodiment of the present invention, there is provided a cell culture method including: a seeding step of seeding a cell onto a culture sheet which is deformable and to which the cell is allowed to adhere; a culture step of culturing the cell; an observation step of observing the cell; and a detachment step of detaching the cell. In at least one of the seeding step, the culture step, the observation step, or the detachment step, a die member is pressed to deform at least a part of the culture sheet.

In the seeding step in the second embodiment, the culture sheet can be deformed such that at least a part of the culture sheet is concave with respect to a culture surface.

Further, in the culture step in the second embodiment, the culture sheet can be deformed such that at least a part of the culture sheet has a shape concave with respect to a culture surface or a shape convex with respect to the culture surface.

In the detachment step in the second embodiment, the culture sheet can be deformed such that at least a part of the culture sheet has a shape convex with respect to the culture surface.

Further, preferably, the shape of at least a part of the culture sheet can be made concave with respect to the culture surface in the seeding step, can be made concave with respect to the culture surface or convex with respect to the culture surface in the culture step, can be made flat in the observation step, and can be made convex with respect to the culture surface in the detachment step.

### EXAMPLE 1

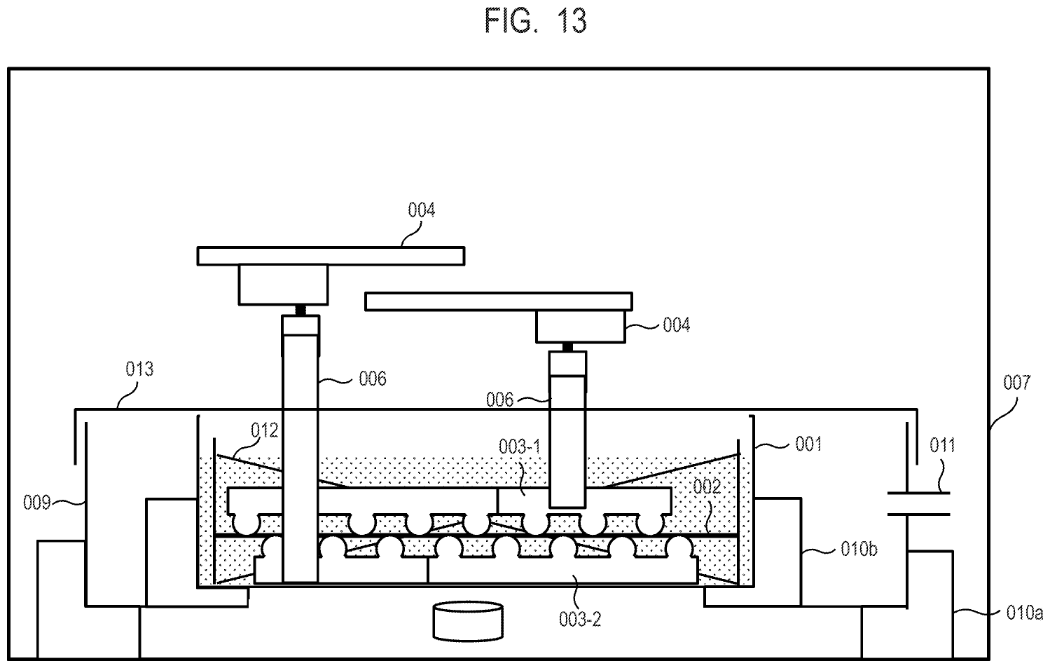

Below, Example 1 is described with reference to FIG. 13. In the drawing, the same components are denoted by the same reference symbols in principle, and description thereof is omitted.

In Example 1, the cell culture system includes a safety cabinet that is the outer vessel 007. In the safety cabinet, a base A 010a is placed, and a chamber vessel 009 made of acryl is placed on the base A 010a. In the chamber vessel 009, the entire upper surface and a central portion of the lower surface are opened, and a lid 013 described later can be placed on the upper surface. In the lower surface, a base B 010b is placed along the opening. In addition, in a side surface, an air duct 011 for supplying hot air, CO₂, and the like from outside is provided. The vessel 001 is a petri dish that is made of polystyrene and does not include a lid. In the vessel, the culture sheet 002 formed of a polycarbonate sheet having a thickness of 0.1 mm or smaller is placed. The culture sheet 002 is fixed to the frame member 012 with a margin large enough for the culture sheet 002 to be deformed when the die member 003 is pressed against the culture sheet 002. The frame member 012 is made of meshed polycarbonate and can be attached to the inside of the vessel. Thus, the culture sheet 002 can be fixed in the vessel. The upper and lower die members (die members 003-1 and 003-2) are each made of fluorocarbon resin and each have a configuration in which rods each having a diameter ϕ of 1 mm are arranged at an interval of 2 mm. Further, the rods are symmetrically placed at the opposite ends of the culture sheet 002 (see FIG. 12A), and the central portion of the culture sheet 002 does not come into contact with the rods even during deformation of the culture sheet 002. In addition, plasma treatment is performed only on the central portion that does not come into contact with the rods on one side (upper side) in the culture sheet 002. This makes it easier for cells to adhere thereto, and contact between cells and the die members 003 is prevented.

The drive unit 004 is prepared for each of the upper and lower die members. For horizontal drive, a linear stage is used, and for vertical drive, an actuator such as a solenoid is used. The drive units 004 and the die members 003 are connected via the arm 006 made of stainless steel, and the arm 006 has a sectional shape that is long in a scanning direction. The lid 013 having a hole bored through only an area where the arm 006 passes is attached to the upper surface of the chamber vessel 009. In this reference example, the range of movement of the linear stage is about several millimeters. Thus, a cushioning member is placed in a gap between the hole and the arm 006, to prevent the gap from being opened widely during movement of the arm, thereby forming a configuration in which gas in the chamber vessel is hard to escape to the outside.

On the lower side of the chamber vessel 009, a lens for a phase-contrast microscope corresponding to the optical-image acquisition unit 005 is placed, and thus cells on the culture sheet 002 can be observed. Further, a monitor for displaying an image is also placed.

An actual procedure for cell culture with the use of this system is described.

### Initial Setting

The base A 010a, the chamber vessel 009, a phase-contrast microscope that is the optical-image acquisition unit 005, and the drive unit 004 are set in a safety cabinet that is the outer vessel 007.

First, the vessel 001 having been sterilized is set in the base B 010b in the chamber vessel, and the second die member 003-2 is put into the vessel 001. The second die member 003-2 is equipped with the arm 006 connectable to the drive unit 004 in advance. Subsequently, the frame member 012 to which the culture sheet 002 is attached is fit into the vessel 001. At that time, the frame member 012 is fit such that the arm 006 is inserted into a gap between the frame member 012 and the culture sheet 002. After the culture sheet 002 is placed in the vessel 001, the arm 006 attached to the second die member 003-2 is connected to the drive unit 004, and the first die member 003-1 is placed in the vessel.

### Cell Culture

The drive unit 004 is driven, and the culture sheet 002 is sandwiched between the first die member 003-1 and the second die member 003-2 and is entirely deformed into a shape with projections and recesses as illustrated in FIG. 3B or FIG. 3C. As a result of this, tension is applied to the culture sheet 002 having been provided with some slack, and the culture sheet 002 is placed at a certain height in the vessel.

Subsequently, a culture medium is injected until the entire culture sheet 002 is immersed in the culture medium. After that, a suspension of cells is seeded onto the culture sheet 002. In Example 1, this operation is performed manually, but a suspension may be injected through a pump or the like after insertion of a tube into the vessel.

After seeding of cells, the lid 013 is put on the upper surface of the chamber vessel 009. The lid 013 is divided into a plurality of parts, and a space is formed only in a movable range of the arm connected to the drive unit 004 while gaps are filled with an elastic rubber material. Because of this elasticity, there is formed a configuration in which it is hard to open a gap despite movement of the arm 006.

A tube is attached to the air duct of the chamber vessel 009 so that hot air including CO₂ can circulate from outside. Consequently, the inside of the chamber can be kept at a CO₂ concentration of about 5% and at a temperature of about 37 degrees, which enables cell culture on the culture sheet 002 having a three-dimensional structure including projections and recesses.

### Change in Substrate Shape and Cell Observation

In observing a state of cells, when the cells are on projections and recesses, it is impossible to provide focus positions over the entire area, which makes it difficult to observe the cells. In this regard, the culture sheet 002 is released from fixing by the die member 003, and thus the projections and the recesses of the culture sheet 002 are moderated. This makes it easier to achieve focusing on the entire area. Note that, in Example 1, only the first die member 003-1 is pressed against the culture sheet 002 at an appropriate pressure, and thus the culture sheet 002 has a flat shape or a substantially flat shape along a surface from which the second die member 003-2 is removed. Consequently, it becomes easier to focus on the cells on the culture sheet 002, to facilitate observation. Based on a result of observation at that time, timings for replacement of a culture medium, injection of a reagent, and detachment of cells are checked.

After the end of the observation, in a case in which cell culture is continued, the culture sheet 002 is again sandwiched between the first die member 003-1 and the second die member 003-2, and thus the culture sheet 002 is returned to a shape with projections and recesses.

As described above, in Example 1, the flexible culture sheet 002 to which cells can adhere to be cultured is compressed and released by the die member 003, and thus is deformed into a shape with projections and recesses or a flat shape. In addition, the culture sheet 002 is caused to have a shape including projections and recesses suitable for mass culture during culture, and is caused to have a flat shape advantageous in focusing during observation of cells, to thereby enable cell culture in a substrate shape suitable for each culture step.

Note that, it is supposed in Example 1 that an iPS cell is used, but almost all adherent cells can be used. For use of those, it is only required to appropriately change kinds of a culture medium and a reagent, an observation timing, the shape of the die member 003 in accordance with the characteristic of each cell and a protocol to be executed.

### EXAMPLE 2

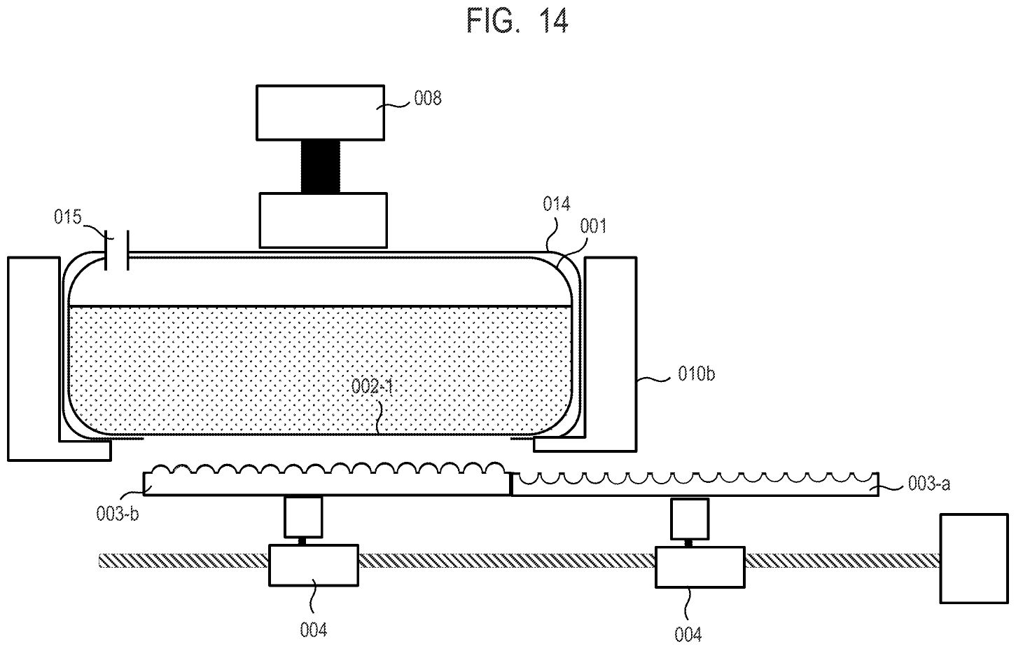

Below, Example 2 is described with reference to FIG. 14. Description of components similar to those in Example 1 in the drawing is omitted.

An outer vessel frame 014 formed of a PET material is placed in the base B 010b. The outer vessel frame 014 has a bottom surface, almost all of which is opened, and surrounds a culture bag 002-1, at least a part of which is formed of a polycarbonate sheet forming the culture sheet 002. The culture bag 002-1 is equipped with a supply/discharge port 015 through which a culture medium, a reagent, and a cell are put in and out.

Below the base B 010b, there are placed a plurality of die members 003 each having a size substantially equal to the size of the opening in the lower surface of the vessel. As the drive unit 004 for driving those die members 003, a pulse motor and a ball screw are used for horizontal drive, and an actuator such as a solenoid is used for vertical drive. The die members 003 include a die member provided with many convex surfaces and a die member provided with many concave surfaces, and those are made of stainless steel. Note that, though not shown, the die member 003 that does not include a recess or a projection but is flat is also included, and this is formed of a transparent glass plate.

The external-force applying unit 008 for pushing the vessel 001 into the lower side is placed above the vessel 001 or the outer vessel frame 014. Further, though not shown, a phase-contrast microscope that is a first optical-image acquisition unit is provided below the vessel, and a camera that is a second optical-image acquisition unit is provided above the vessel. Thus, cells on the culture sheet 002 can be observed. With the phase-contrast microscope, a detailed structure of a cell can be checked, and with the camera, a rough size of a colony can be checked. Further, a monitor for displaying images of a cell and a colony is also placed.

An actual procedure for cell culture is described.

### Initial Setting

First, as the vessel 001, a configuration is prepared in which the culture bag 002-1, at least a part of which is formed of the culture sheet 002 made of polycarbonate, is covered with the outer vessel frame 014 that includes an opened bottom surface and the opened supply/discharge port 015 and is made of PET. The thus prepared vessel 001 is placed in the base B 010b. After that, a tube is attached to the supply/discharge port 015.

### Cell Culture

A culture medium is supplied from the supply/discharge port 015 to the vessel through the tube. Subsequently, the die member 003 provided with many recesses is moved to the opening in the bottom surface of the outer vessel frame 014, and is pressed against the culture sheet 002. Further, the entire vessel is pressed against the die member 003 by the external-force applying unit 008 for improved close contact, and thus many recesses are formed in the culture sheet 002 as illustrated in FIG. 10C. In this state, a suspension of cells is injected through the tube. After that, CO₂ is supplied from outside through the tube, and the CO₂ concentration in the inside is kept at about 5%. Further, the base B 010b and the die member 003 are each given with a heater function, and keep the temperature of the culture medium at about 37 degrees. With this configuration, cells can be cultured on the culture sheet 002 having a three-dimensional structure including concave and convex surfaces.

### Change in Substrate Shape and Cell Observation

In observing a state of cells, the drive unit 004 is driven to change a die member 003-a including recesses to a die member of flat glass (not shown). Then, the die member is pressed, to thereby flatten the culture sheet 002 for the cells, and observation is performed with the use of the phase-contrast microscope. After that, the die member is changed to the die member 003-a including recesses, and thus cell culture on the concave surface can be continued again.

Further, just for rough check of a colony size or the like, it is also possible to make determination as to whether the check is performed with the use of the upper camera and the culture is maintained while leaving the die member 003-a including the recesses placed there, or the die member 003-a is changed to another die member 003.

### Change in Substrate Shape and Cell Detachment

After culture and observation are repeated and it is checked that a colony has been appropriately formed, detachment of cells is performed. At that time, the die member is changed to a die member 003-b including projections, which is then pressed against the culture sheet 002 such that a portion to which cells are adhering becomes convex. Then, the portion to which the cells are adhering is enlarged. In this state, an enzyme for cell detachment is injected. In Example 2, a trypsin solution is injected. Thus, by making the portion to which the cells are adhering in the culture sheet 002 convex, it is possible to allow trypsin to easily come into contact with the cells, and hence, facilitation of cell detachment is expected.

As described above, in Example 2, the different die members 003 (003-b, 003-a) that include projections, recesses, and a flat surface, respectively, are pressed against the culture bag 002-1 formed of the flexible culture sheet 002 to which cells can adhere and be cultured. This enables cell culture in a substrate having a shape suitable for each culture step.

Specifically, Example 2 described above shows two examples as follows. That is, in Example 2-1, in performing culture, the culture sheet is made concave with respect to the culture surface during culture, is made flat during observation, and is made convex with respect to the culture surface during detachment. As Example 2-2, in performing culture, the culture sheet is made concave with respect to the culture surface during culture and observation and is made convex with respect to the culture surface during detachment.

Note that, it is supposed in Example 2 that an iPS cell is used, but almost all adherent cells can be used. In this regard, it is only required to appropriately change kinds of a culture medium and a reagent, an observation timing, and the shape of the die member 003 in accordance with the characteristic of each cell and a protocol to be executed.

The present invention provides a cell culture system and a cell culture method for deforming a culture sheet into a shape suitable for each step of cell culture. In each step, the shape of the culture sheet is optimized, and hence, the efficiency of seeding, the accuracy in observation, and the efficiency of detachment can be enhanced.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-117226, filed Jul. 22, 2022, which is hereby incorporated by reference herein in its entirety.

What is claimed is:

1. A cell culture system comprising:
a culture sheet which is deformable, and to which a cell is allowed to adhere;
a die member configured to be pressed against the culture sheet to deform the culture sheet;
a drive unit configured to drive the die member; and
an optical-image acquisition unit configured to acquire an optical image of the cell.

2. The cell culture system according to claim 1, further comprising a vessel for accommodating a culture medium and the culture sheet.

3. The cell culture system according to claim 2,
wherein the die member includes two or more die members including a first die member and a second die member,
wherein the first die member is configured to be pressed against a culture surface of the culture sheet, and
wherein the second die member is configured to be pressed against a back surface opposite to the culture surface of the culture sheet.

4. The cell culture system according to claim 1, further comprising a vessel for accommodating a culture medium,
wherein the culture sheet forms a part of the vessel.

5. The cell culture system according to claim 4,
wherein the die member includes two or more die members including a first die member and a second die member,
wherein the first die member is configured to be pressed against a culture surface of the culture sheet, and
wherein the second die member is configured to be pressed against a back surface opposite to the culture surface of the culture sheet.

6. The cell culture system according to claim 1, wherein the die member includes a plurality of parts.

7. The cell culture system according to claim 1, wherein the die member is prevented from coming into contact with a part of an area to which the cell is allowed to adhere in a culture surface of the culture sheet.

8. The cell culture system according to claim 1, wherein the drive unit includes an arm.

9. The cell culture system according to claim 2, further comprising an outer vessel for accommodating at least the vessel, the culture sheet, the die member, and the drive unit.

10. The cell culture system according to claim 1, further comprising an external-force applying unit that is distinct from the drive unit and is configured to press the die member against the culture sheet.

11. The cell culture system according to claim 2, wherein an external-force applying unit is configured to increase and reduce a pressure in the vessel.

12. The cell culture system according to claim 1, further comprising a drive control unit configured to control the drive unit based on the optical image of the cell such that at least a part of the culture sheet has any of a flat shape, a shape concave with respect to a culture surface, and a shape convex with respect to the culture surface.

13. A cell culture method comprising:
seeding a cell onto a culture sheet which is deformable and to which the cell is allowed to adhere;
culturing the cell;
observing the cell; and
detaching the cell,
wherein at least one of the seeding, the culturing, the observing, or the detaching includes pressing a die member to deform at least a part of the culture sheet.

14. The cell culture method according to claim 13, wherein the seeding includes deforming the culture sheet such that at least a part of the culture sheet is concave with respect to a culture surface.

15. The cell culture method according to claim 13, wherein the culturing includes deforming the culture sheet such that at least a part of the culture sheet has one of a shape concave with respect to a culture surface or a shape convex with respect to the culture surface.

16. The cell culture method according to claim 13, wherein the detaching includes deforming the culture sheet such that at least a part of the culture sheet has a shape convex with respect to a culture surface.

17. The cell culture method according to claim 13, wherein the culture sheet is deformed such that:
at least a part of the culture sheet is concave with respect to a culture surface in the seeding;
at least a part of the culture sheet has one of a shape concave with respect to the culture surface or a shape convex with respect to the culture surface in the culturing;
at least a part of the culture sheet is flat in the observing; and
at least a part of the culture sheet is convex with respect to the culture surface in the detaching.
